(19) United States
(12) Patent Application Publication
(10) Pub. No.: US 2005/0085733 A1
(43) Pub. Date: Apr. 21, 2005

(54) Ear thermometer illumination system

(76) Inventor: Anthony Wong, Franklin, MA (US)

Correspondence Address: BARNES & THORNBURG, 11 SOUTH MERIDIAN, INDIANAPOLIS, IN, 46204, US

(21) Appl. No.: 10/688,334

(22) Filed: Oct. 17, 2003

(51) Int. Cl. A61B 6/00

(52) U.S. Cl. 600/474; 600/549

(57) ABSTRACT

An infrared ear thermometer is provided to read the temperature of the tympanic membrane within the ear canal of a patient. The infrared ear thermometer includes a housing, a probe coupled to the housing, and an infrared sensor received at least in part within the probe.

### BACKGROUND

The present disclosure relates to a thermometer, and particularly to an infrared ear thermometer.

Infrared ear thermometers include a probe that is inserted into the ear canal of a patient. An infrared sensor within the probe measures the body temperature of the patient by sensing the intensity of an infrared ray radiated from within the ear canal of a patient.

### SUMMARY

According to the present disclosure, an infrared ear thermometer is provided to read the temperature of the tympanic membrane within the ear canal of a patient. The thermometer includes a housing, a probe coupled to the housing, and an infrared sensor received at least in part within the probe. A light of the thermometer is positioned adjacent the probe to illuminate an area around the probe when the infrared sensor is activated by a user. The light is provided to illuminate an ear canal of a patient as the probe is being inserted into the ear canal prior to reading the temperature of the patient.

Illustratively, the light is a light-emitting diode.

Further illustratively, the thermometer includes a light cover coupled to the housing and the probe. The light illuminates the light cover when the infrared sensor is activated by a user. The illustrative light cover is ring-shaped and at least a portion of the probe is received through an aperture of the ring-shaped light cover. The illustrative light cover is substantially translucent to allow the light to shine through the light cover and illuminate an area surrounding the probe, specifically, the ear canal of a patient.

Further according to the present disclosure, a method for reading the temperature of a patient using an infrared ear thermometer is provided. The method comprises the acts of illuminating an area surrounding a probe of an infrared ear thermometer and inserting the probe into an ear canal of a patient to illuminate the ear canal of the patient. The method further comprises the act of sensing the temperature of a tympanic membrane of the patient within the ear canal of the patient. Illuminating the area surrounding the probe includes the act of activating a light-emitting diode of the thermometer adjacent the probe.

The method further comprises the acts of flashing a light a plurality of times to and producing an audible alarm to indicate that the temperature of the tympanic membrane has been sensed. Thus, both audible and visual indicators are provided to inform the user that the temperature has been sensed and the probe can be removed from within the patient's ear canal.

Additional features of the present disclosure will become apparent to those skilled in the art upon consideration of the following detailed description of preferred embodiments exemplifying the best mode of carrying out the disclosure as presently perceived.

### BRIEF DESCRIPTION OF THE DRAWINGS

The detailed description particularly refers to the accompany figures in which:

FIG. 1 is a rear perspective view of an infrared ear thermometer of the present disclosure showing the ear thermometer including a housing, a probe coupled to the housing, and a storage unit coupled to a bottom end of the housing for stowing disposable probe covers (shown in FIG. 2) therein;

FIG. 2 is a side view of the ear thermometer of FIG. 1 showing a light halo coupled to and positioned between the housing and the probe for illuminating the ear area during use to provide better visibility when a user is placing the probe of the thermometer within the ear of the patient to take the temperature of the patient, for example;

FIG. 3 is a rear perspective exploded assembly view of the ear thermometer showing a front and rear housing portion of the housing, the light halo, the storage unit, and a probe-cover dispenser for storing the disposable probe covers therein;

FIG. 3a is a perspective view of a portion of the housing and the probe cover storage unit detached from the housing showing snaps or fastener portions of the housing and corresponding snaps or fastener portions of the storage unit to allow a user to removably couple the storage unit to the housing;

FIG. 4 is a perspective view of the ear thermometer showing the storage unit removed from the ear thermometer and showing the probe-cover dispenser having been removed from within a cavity of the probe cover storage unit in order to allow a user to remove one or more disposable probe covers from within the dispenser;

FIG. 5 is a perspective view of the storage unit of the ear thermometer and the probe-cover dispenser showing the probe cover storage unit turned upside down to expose an aperture formed in a bottom wall of the storage unit;

FIG. 6 is a perspective view of the probe-cover dispenser showing a user pulling a single disposable probe cover from within the dispenser and tearing the probe cover along a perforated edge to separate the probe cover from the other probe covers contained within the dispenser;

FIG. 7 is a perspective view of the of probe cover storage unit in the upside down or loading position showing the disposable probe cover positioned over the aperture of the storage unit to rest within an outer lip of the storage unit;

FIG. 8 is a perspective view similar to FIG. 7 showing a user having inserted the probe of the ear thermometer through the aperture of the storage unit to engage a plastic wrap portion of the disposable cover; and

FIG. 9 is a perspective view of a portion of the ear thermometer showing the disposable cover wrapped around and covering the probe of the ear thermometer for use with a patient.

### DETAILED DESCRIPTION

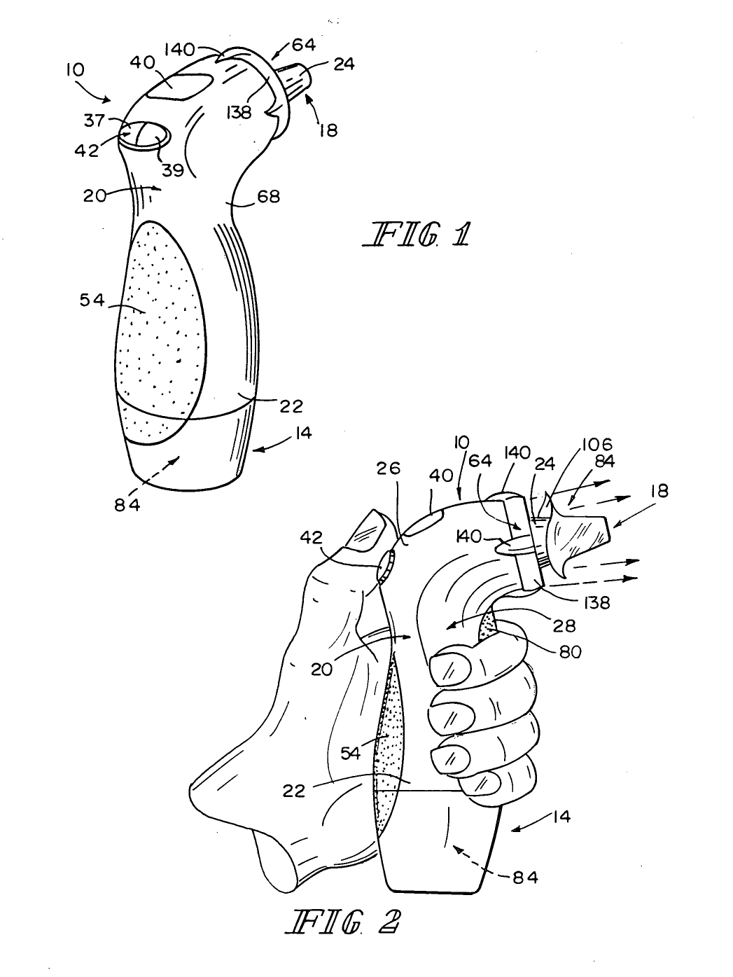

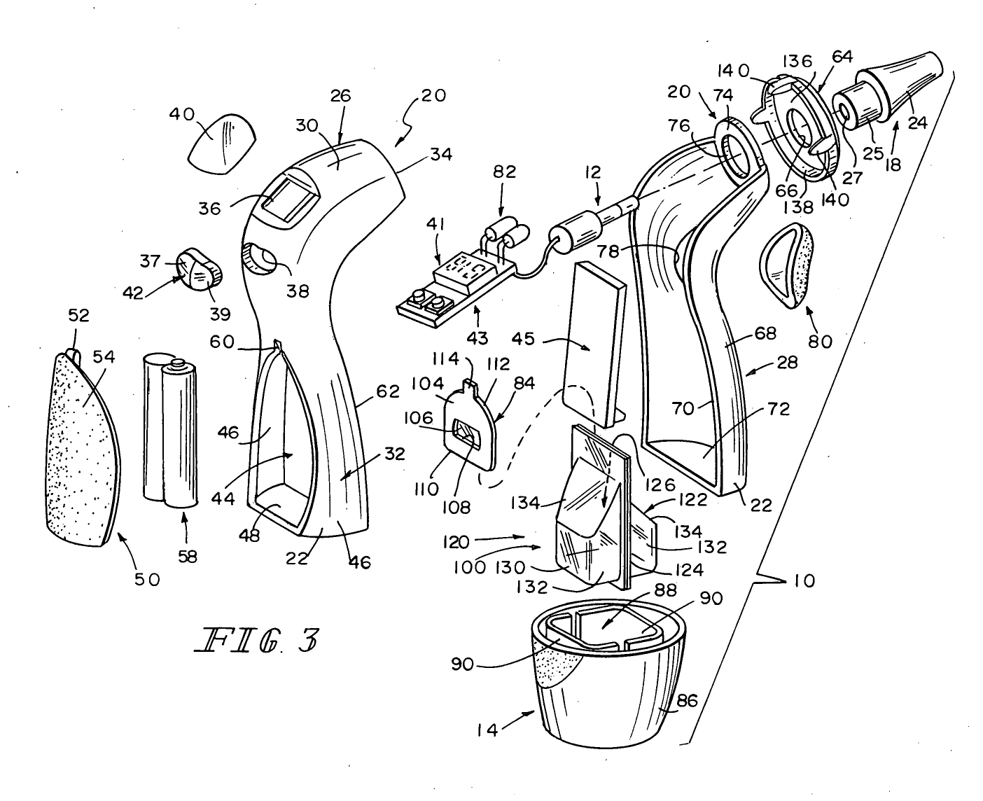

An infrared ear thermometer 10, as shown in FIG. 1, is provided to measure the temperature within an ear canal (not shown) of a patient by using an infrared sensor 12 (FIG. 3) located within a probe 18 of thermometer 10. Such an ear thermometer is shown and described in a U.S. patent application titled PROBE COVER STORAGE SYSTEM FOR EAR THERMOMETER filed concurrently herewith, the disclosure of which is hereby incorporated by reference herein. Infrared ear thermometer 10 further includes a light halo or light cover 64 to illuminate an area surrounding the probe 18 when a user is inserting the probe 18 into the ear canal of a patient, for example. As is described in further detail below, light halo 64 is ring-shaped and is illuminated by a pair of light-emitting diodes (LEDs) 82 of thermometer 10 which are activated when thermometer 10 is turned “on”.

Infrared ear thermometer further includes a storage unit 14 to stow disposable probe covers 84, as suggested in FIG. 4. As is mentioned in greater detail below, disposable probe covers 84 are provided to cover probe 18 of the thermometer 10, as shown in FIG. 2, prior to inserting the probe 18 into the ear canal of a patient. Stowing disposable probe covers 84 within storage unit 14 of infrared ear thermometer 10 helps to prevent a caregiver from misplacing the probe covers 84.

As shown in FIG. 1, infrared ear thermometer 10 includes a main housing 20, probe cover storage unit 14 coupled to a bottom end 22 of housing 20, and probe 18 also coupled to housing 20. Infrared sensor 12 (shown in FIG. 3) is positioned, at lease in part, within probe 18. In operation, sensor 12 picks up the eardrum or tympanic membrane's radiant heat energy. Microelectronics associated with a display printer control board 43 and a main printer control board 45 (discussed below) determine the eardrum temperature from the infrared sensor's electrical output. A removable probe cap (not shown) is provided for covering probe 18 when thermometer 10 is not in use.

Illustratively, housing 20 is ergonomically designed to provide for a “pistol grip” gripping style, as shown in FIG. 2. This ergonomic gripping style aides in preventing inaccurate temperature recording methods by helping the caregiver position the probe 18 properly within the ear canal of the patient, for example. As shown in FIG. 2, housing 20 acts as a handle portion of thermometer 10 for a user to grasp while operating thermometer 10. Although not discussed in detail herein, it is commonly understood that the infrared thermometer operates by detecting an infrared, or heat, ray radiated from the eardrum, specifically the tympanic membrane, and its vicinity.

As shown in FIG. 3, housing 20 includes a front housing portion 26 and a rear housing portion 28 coupled to the front housing portion 26. Front housing portion 28 is a monolithic member and includes a top, curved head portion 30 and a substantially vertical body portion 32 integrally coupled to the head portion 30. The curved head portion 30 is formed to define a curved end surface 34, a display aperture 36, and a power-button aperture 38.

An illustrative display window 40 of the thermometer 10 is positioned within display aperture 36 to cover a liquid crystal display (LCD) 41 which is provided to display digitally the temperature of the patient as read by the infrared sensor 12. Although an LCD is provided, it is within the scope of this disclosure to provide a thermometer 10 having other suitable displays such as a light-emitting diode (LED), for example. Illustrative LCD 41 is coupled to a display printer circuit board (PCB) 43 and other electrical components, such as for example, a power momentary switch and a mute momentary switch, of thermometer 10 which are housed within housing 20 and which connect the infrared sensor 12 with LCD 41.

A main printer circuit board (PCB) 45 is also housed within housing 20 of thermometer 10. Thermometer 10 also includes other components in communication with main PCB 45 such as an infrared momentary switch (not shown), a speaker (not shown), and lead contacts (not shown) for batteries 58. Main PCB 45 also communicates with display PCB 43.

An on/off and mute power button 42 of thermometer 10 is positioned within power-button aperture 38. A left portion 37 of button 42 is provided to turn the power on and off when depressed by a user while a right portion 39 is provided to mute or turn off an audible beep sounded by the thermometer when the patient's temperature has been recorded. As shown in FIG. 2, for example, the ergonomic pistol-grip style provides that the user's thumb engage the power button 42 in order to activate and deactivate the power button 42. Power button 42 is similarly in communication with internal electrical components of thermometer 10.

Body portion 32 of front housing portion 26 includes an aperture 44 defined by opposing side walls 46 spaced-apart from each other and a bottom wall 48 coupled to each of the two opposing side walls 46. A removable battery door 50 of body portion 32 includes a tab 52 and a main body 54 coupled to tab 52. Battery door 50 is provided to cover aperture 44 to maintain batteries 58 of thermometer 10 within housing 20. Illustratively, batteries 58 are AAA batteries and are housed between side walls 46 of body portion 32 of front housing portion 26. Tab 52 of battery door 50 is received within a notch 60 of body portion 32. A caregiver may remove battery door 50 from body portion 32 to replace the batteries 58 of thermometer 10 as needed.

Rear housing portion 28 of housing 20 includes a curved wall 68 defining an edge 70 coupled to a curved edge 62 of front housing portion 26. Rear housing portion 28 further includes a bottom wall 72 coupled to curved wall 68 and a face plate 74 coupled to curved wall 68 and formed to define an aperture 76 for receiving a portion of the infrared sensor 12 and probe 18 therethrough.

Illustrative probe 18 includes a substantially cone-shaped probe body 24 coupled to an anchor body 25. The tapered or cone-shaped probe body 24 provides for ease of insertion of the probe 18 into the ear canal of a patient. A passageway 27 is formed through both probe body 24 and anchor body 25 to receive at least a portion of infrared sensor 12 therein. An infrared-button aperture 78 is formed in curved wall 68, as shown in FIG. 3, to receive an infrared button 80 therein. Infrared button 80 is a push button for selective actuation of the infrared sensor 12. By depressing push button 80, as shown in FIG. 2, a user is able to activate the infrared sensor 12 to take the temperature of a patient, for example.

A light halo or ring 64 is coupled to housing 20 and is formed to include an aperture 66 aligned with aperture 76 of rear housing portion 28. Similar to aperture 66, aperture 76 is formed to receive a portion of probe 18 therethorough. Light halo 64 is in communication with bright amber light-emitting diodes (LEDs) 82 coupled to display PCB 43. The amber LEDs 82 are provided to illuminate halo 64, as shown in FIG. 2, when a user activates the left portion 37 of power button 42. Thus, light halo 64 is illustratively used as a light cover. The illuminated halo 64 provides illumination for a user when probe 18 is inserted into the ear canal of a patient to take the temperature therein. Illustratively, halo 64 is made of a translucent or semi-translucent material, such as plastic, to allow the light from LEDs 82 to illuminate halo 64 and thus illuminate the area surrounding halo 64 and probe 18. Illustrative halo 64 includes a front, circular wall 136 having aperture 66 formed therethrough, a sidewall 138 coupled to front wall 136 and three illustrative tabs 140. Front wall 136 lies adjacent face plank 74 so that aperture 76 of face plate 74 with aperture 66 of halo 64 to receive anchor portion 25 of probe 18 therein. LEDs 82 are positioned to be near or adjacent halo 64 in order to illuminate halo when activated.

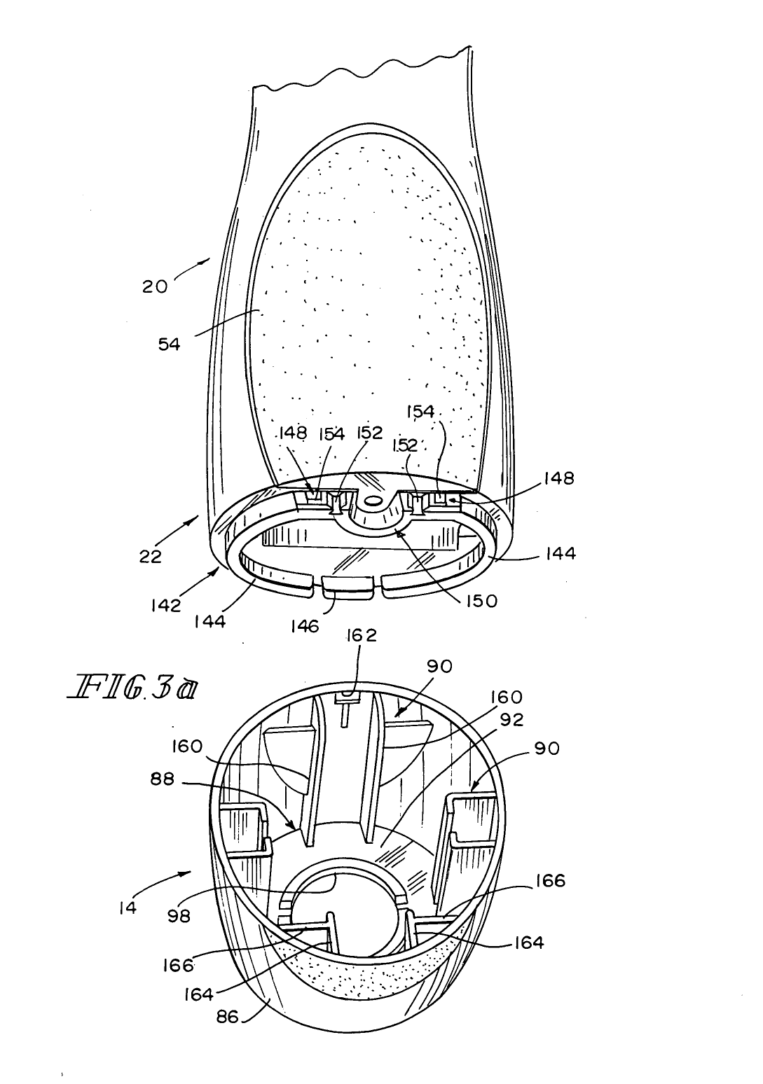

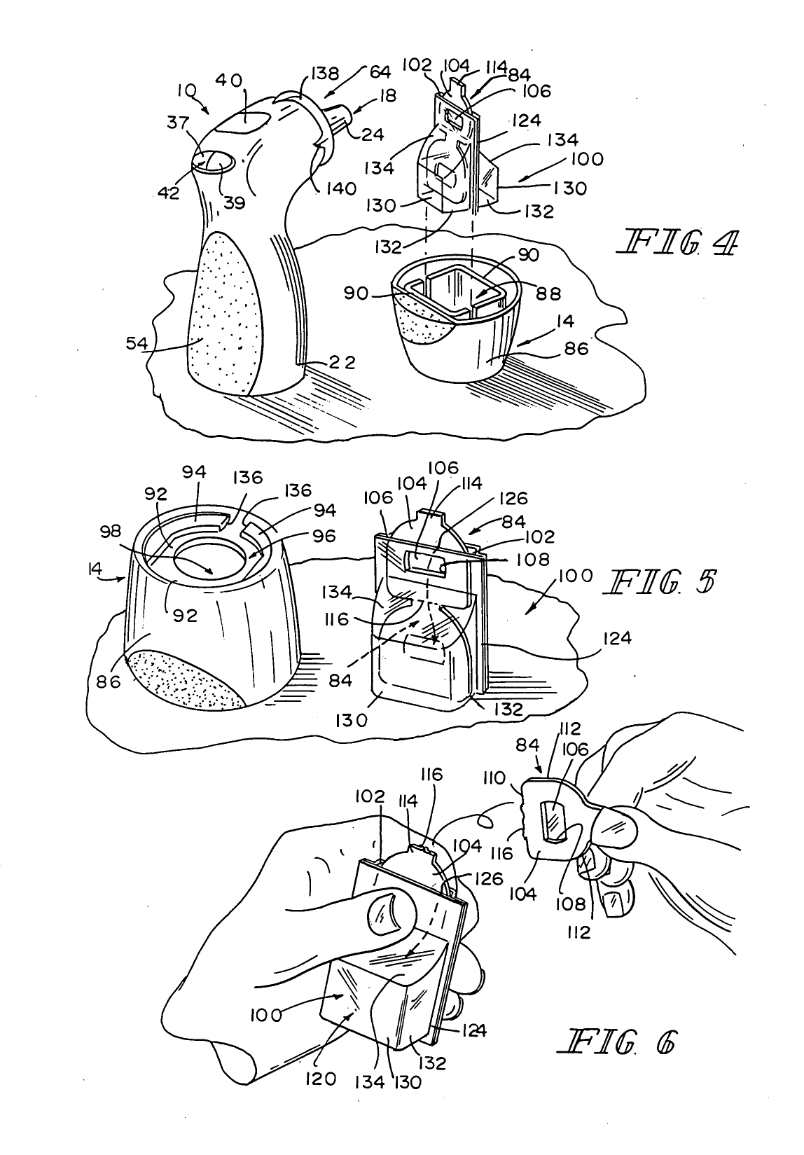

Looking now to FIGS. 3-5, 7, and 8, thermometer 10 further includes probe cover storage unit 14, as mentioned above, coupled to bottom portion 22 of housing 20. Storage unit 14 is removable from housing 20 by the user. As is discussed in greater detail below, the probe cover storage unit 14 is provided to store disposable probe covers 84, as shown in FIGS. 5 and 6, for example.

Probe cover storage unit 14 includes an outer curved wall 86 formed to define a cavity 88 for receiving and storing probe covers 84 therein. Illustratively, outer curved wall 86 is generally cylindrical. Inner wall portions 90 of unit 14 are coupled to outer curved wall 86 and define an inner portion of cavity 88. As shown in FIG. 5, unit 14 further includes a bottom wall 92 and an outer lip or rim 94 coupled to bottom wall 92 to define a probe cover-receiving area 96. Bottom wall 92 is coupled to outer curved wall 86 and is formed to define an aperture 98 therethrough for communication with cavity 88. As is discussed in greater detail below, the bottom wall 92 and outer rim 94 cooperate to define a probe cover applicator of the storage unit 14 to aide a user in placing the disposable probe cover 84 properly onto the probe body 24 prior to reading the temperature of a patient.

Illustratively, probe covers 84 are stored within a probe-cover dispenser 100, shown in FIGS. 3-6. Dispenser 100 is stored normally within cavity 88 of storage unit 14. Dispenser 100 includes a top opening 102 to allow a user to retrieve the probe covers 84 stored within. Illustrative dispenser 100 includes two body halves 120, 122 coupled to each other to form an outer seam 124. Top opening 102 is formed through a portion of seam 124 to communicate with an inner cavity 126 formed to receive and store the probe covers 84. Each body half 120, 122 includes a bottom wall 128, a front wall 130, opposite side walls 132, and an inclined top wall 134. Top opening 102 is formed through the portion of the seam 124 adjoining the top wall 134 of each body half 120, 122. Cavity 126 is defined by bottom, front, top, and opposite side walls 128, 130, 134, 132 of each body half 120, 122 of dispenser 100.

The body halves 120, 122 of dispenser 100 cooperate to define a dispenser having a bottom wall (defined by the bottom wall 128 of each body half 120, 122), four side walls (defined by the front and opposite side walls 130, 132 of each body half 120, 122), and a top wall (defined by the top wall 134 of each body half 120, 122). It is within the scope of this disclosure to provide a thermometer 10 having a probe-cover dispenser having another suitable shape for dispensing the probe covers 84 or other suitable probe covers therefrom. Illustrative dispenser 100 is also substantially clear or transparent to allow the user to view the probe covers 84 within cavity 126.

Each illustrative probe cover 84 is substantially planar and includes an outer portion 104 and an inner portion 106 coupled to outer portion 104 and positioned within an aperture 108 of outer portion 104. Illustratively, outer portion 104 is made of a paper material and inner portion 106 is made of a generally clear, thin, pliable material such as polyethylene or a type of plastic wrap, for example. Illustrative probe covers 84 stowed within thermometer 10 are Ear Thermometer Disposable Probe Covers, model number 49006, manufactured by Microlife USA, Inc. of Biddeford, Me. Thermometer 10 may include other suitable probe covers 84 which may be stowed in a storage unit and/or a dispenser of the thermometer 10, such as illustrative storage unit 14 and probe-cover dispenser 100. For example, U.S. Pat. No. 6,123,454, discloses similar such probe covers, the disclosure of which is hereby incorporated by reference herein.

Each illustrative probe cover 84 is substantially “D-shaped” and includes a straight edge 110 and a curved edge 112. A tab portion 114 of each probe cover 84 is coupled to curved edge 112. As shown in FIGS. 5 and 6, tab portion 114 of each probe cover 84 is coupled to the straight edge 110 of an adjacent probe cover 84 along a perforated line 116. Thus, multiple probe covers 84, which are coupled to each other, are stored within probe-cover dispenser 100. A single probe cover 84 is threaded through top slot or opening 102 of dispenser 100, as shown in FIG. 5, to be grasped by the user. Once grasped by the user, the exposed probe cover 84 is pulled substantially out of dispenser 100 and torn from the adjacent probe cover 84 along perforated line 116, as shown in FIG. 6.

Each probe cover 84 is formed to cooperate with the shape of the probe cover-receiving area 96 of the probe cover storage unit 14. As shown in FIGS. 5 and 7, rims 94 define a substantially D-shaped cavity a probe cover receiving area 96 to receive a single probe cover 84 therein. At least a portion of outer portion 104 of the probe cover 84 rests on an outer surface 136 of end wall 92. Edges 110, 112 of the particular probe cover placed within probe cover-receiving area 96 substantially abut or lie adjacent to outer lip 94, as shown in FIG. 7.

In order to cover probe 18 with a disposable probe cover 84, a user or caregiver first removes probe cover storage unit 14 from housing 20. Storage unit 14 may be coupled to housing 20 by various suitable coupling means including, but not limited to, snap-on type detents and notches, threaded portions, a single hinged side, screws, or other suitable means. Storage unit 14 is thus coupled to housing 20 in such as way as to allow a user to remove storage unit 14 therefrom to provide access to the probe covers 84 stored therein.

As shown in FIG. 3a, end 22 of illustrative housing 20 includes a rim 142 protruding away from end 22. Rim 142 includes two “C-shaped” rim sections 144, a tab section 146 between one of the ends of the C-shaped rim sections 144, and a first and second snap sections 148 coupled to and positioned between the opposite ends of the C-shaped rim sections 144. A substantially “U-shaped” detent 150 is coupled to and positioned between first and second snap sections 148 as also shown in FIG. 3a. Each of the first and second snap sections 148 includes a notch 152 formed therein and a hook 154 adjacent each notch 152.

As mentioned above, probe cover storage unit 14 includes inner wall portions 90 positioned within cavity 88 and coupled to outer wall 86. Inner wall portions 90 include first and second vertical wall portions 160 coupled to outer wall 86 and spaced-apart from each other. A notch 162 is formed in outer wall 86 and is positioned between the two vertical wall portions 160. When assembled, tab section 146 of housing 20 is positioned between wall portions 160. A detent or hook (not shown) coupled to an outer side (not shown) of tab section 146 is received within notch 162 of storage unit 14 to lock or snap storage unit 14 to housing 20.

Inner wall portions 90 of storage unit 14 further include third and fourth vertical wall portions 164 positioned to lie opposite from first and second vertical wall portions 160. Vertical wall portions 164 are coupled to outer wall 86 and are positioned to lie in space-apart relation to each other. A horizontal wall portion 166 is coupled to each of the third and fourth vertical wall portions 164 at one end and to outer wall 86 at another end, as shown in FIG. 3a. When assembled, U-shaped detent 150 is positioned between third and fourth vertical wall portions 164 while notch 152 of each snap section 148 receives a portion of a respective one of the third and fourth wall portions 164 therein and hook 154 of each snap section 148 receives a portion of a respective one of the horizontal wall portions 166 to couple or snap the storage unit 14 to the housing 20. Storage unit 14 and housing 20 are thus snap-fit to each other.

A user removes illustrative storage unit 14 from housing 20 by pulling storage unit 14 away from housing 20 with sufficient force to un-snap storage unit 14 from housing 20. To reattach storage unit 14 and housing 20, a user aligns the appropriate corresponding components such as notch 162 with tab section 146 and hooks and notches 154, 152 with third and fourth vertical wall portions 164 and horizontal wall portion 166. As mentioned above, although thermometer 10 includes housing 20 and probe cover 14 which are snap-fit to each other, it is also within the scope of this disclosure to include other means for coupling storage unit 14 to housing 20 such as, but not limited to, threaded couplings, hinged couplings, screws, or other suitable means.

Looking to FIG. 4, once storage unit 14 is removed from housing 20, the user then removes the dispenser 100 from within the cavity 88 of storage unit 14. Storage unit 14 is then turned upside-down so that bottom wall 92 and probe cover applicator is accessible to the user, as shown in FIG. 5. Looking now to FIG. 6, the user removes a single disposable probe cover 84 from within dispenser 100 and places the probe cover 84 within cavity 96 of storage unit 14 to rest on bottom wall 92, as shown in FIG. 7, so that probe cover 84 and storage unit 14 are in a loading position ready to receive probe 18 of thermometer 10. As shown in FIG. 7, tab portion 114 of probe cover 84 is positioned between an end wall 135 of each rim 94. Inner portion 106 of probe cover 84 is substantially in alignment with aperture 98 of storage unit 14. Although probe cover 84 and area 96 are each D-shaped, it is within the scope of this disclosure for probe covers 84 to have any suitable shape which corresponds with a suitable shape of area 96 for receiving a probe cover therein.

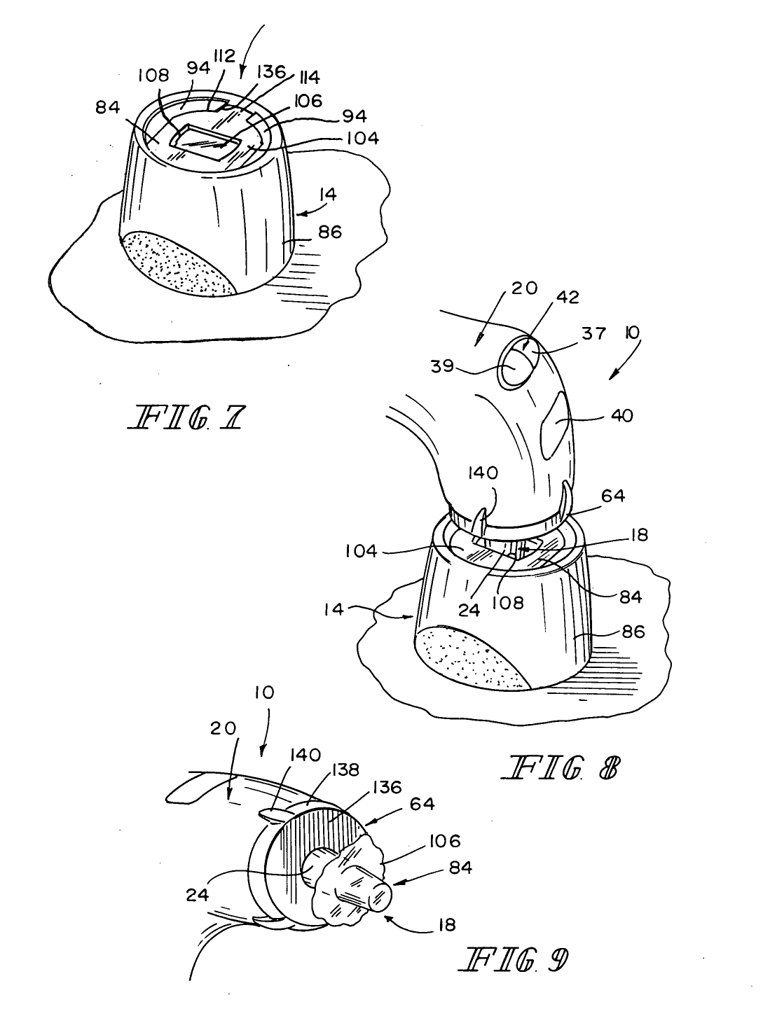

Next, as shown in FIG. 8, the user extends probe 18 of thermometer 10 through aperture 108 of probe cover 84 and through aperture 98 of storage unit 14. This action causes the inner portion 106 of probe cover 84 to be placed around at least a portion of probe 18. Inner portion 106 may also be caused to stretch as probe 18 is inserted through apertures 108 and 98. The thin, plastic material of inner portion 106 sticks to the probe 18 such that when probe 18 is removed from apertures 108 and 98, inner portion 106 remains coupled to and covering at least a portion of the end probe body 24 of probe 18, as shown in FIG. 9. Once disposable probe cover 84 (or at least inner portion 106 of probe cover 84) has been attached probe 18 to substantially cover probe 18, the thermometer 10 is ready to be used to take the temperature of a patient.

In use, as shown in FIG. 2, the user grips thermometer 10 using an ergonomic pistol-grip gripping style where the user's thumb is aligned with the on/off and mute button 42 and the user's forefinger is aligned with infrared button 80. As mentioned above, the user depresses the on/off button 42 to activate the thermometer 10. By turning the thermometer 10 power “on,” the LCD display 41 lights up. The LED's 82 within light halo 64 are also illuminated when the user depresses on/off button 42. The light emitted by the LED's 82 through light halo 64 illuminates the ear and ear canal of the patient to aide the user in low light situations, for example, in positioning the probe 18 properly within the ear canal to obtain the most accurate temperature reading. Once the thermometer 10 is activated, the user then positions the covered probe 18 into the ear (not shown) of a patient to position the probe 18 in alignment with the ear canal of the patient. By depressing infrared button 80, the user activates the infrared sensor 12 within probe 18 to sense the temperature within the ear canal. Specifically, the hottest temperature of the tympanic membrane within the ear canal is located and read by the infrared sensor 12. Once the infrared sensor 12 has sensed and recorded the highest temperature of the tympanic membrane of the patient, the user will hear an audible “beep” or alarm and the LEDs 82 will illustratively flash five time to provide the user with an audible and visual feedback that the temperature has been recorded. The temperature is then displayed digitally on the LCD 41 and viewed by the user through the display window 40.

Once the user has finished taking the temperature of a patient, for example, the probe cover 84 covering probe 18 may be discarded. Probe-cover dispenser 100 may be reinserted into cavity 88 of storage unit 14 and storage unit 14 may be reattached to housing 20. Probe covers 84 need not be kept separate from thermometer 10 or from a the housing 20 of thermometer 10. Thermometer 10, therefore, allows a user to store probe covers 84 together with the temperature sensing portions of thermometer 10 contained within housing 20 as a single device while thermometer 10 is not in use.

1. An infrared ear thermometer comprising
a housing,
a probe coupled to the housing,
an infrared sensor received at least in part within the probe, and
a light adjacent the probe to illuminate an area around the probe when the infrared sensor is activated by a user.

2. The infrared ear thermometer of claim 1, wherein the light is a light-emitting diode.

3. The infrared ear thermometer of claim 1, further comprising a light cover coupled to the housing and the probe and wherein the light is arranged to illuminate the light cover when the infrared sensor is activated by a user.

4. The infrared ear thermometer of claim 3, wherein the light cover is ring-shaped and at least a portion of the probe is received through an aperture formed in the ring-shaped light cover.

5. The infrared ear thermometer of claim 3, wherein the light cover is substantially translucent to allow the light to shine through the light cover and illuminate an area surrounding the probe.

6. An infrared ear thermometer comprising
an infrared sensor housed within a probe configured to be inserted into the ear canal of a patient, the probe being substantially cone-shaped,
a light cover coupled to the probe and positioned to encircle the probe, and
a light adjacent the light cover to shine through the light cover and illuminate an area surrounding the probe when a user places the probe within the ear canal of the patient.

7. The infrared ear thermometer of claim 6, wherein the light cover is ring-shaped and an aperture formed in the light cover receives at least a portion of the probe therein.

8. The infrared ear thermometer of claim 7, wherein the light cover is made of a substantially translucent material.

9. The infrared ear thermometer of claim 7, further comprising a housing of thermometer formed to store electronics of the thermometer therein, and wherein the light cover is coupled to the housing.

10. The infrared ear thermometer of claim 6, wherein the light is a light-emitting diode.

11. The infrared ear thermometer of claim 10, further comprising an on/off button and the light-emitting diode is electrically coupled to the on/off button to be activated by on/off button.

12. A method of reading the temperature of a patient using an infrared ear thermometer, the method comprising the acts of
illuminating an area surrounding a probe of an infrared ear thermometer,
inserting the probe into an ear canal of a patient to illuminate the ear canal of the patient, and
sensing the temperature of a tympanic membrane of the patient within the ear canal of the patient.

13. The method of claim 12, further comprising the act of flashing a light a plurality of times to indicate that the temperature of the tympanic membrane has been sensed.

14. The method of claim 13, further comprising the act of producing an audible alarm to indicate that the temperature of the tympanic membrane has been sensed.

15. The method of claim 12, wherein the illuminating act comprises the act of activating a light-emitting diode adjacent the probe.

16. The method of claim 15, wherein the illuminating act comprises the act of illuminating a ring-shaped light cover around the probe.
